(19) United States
(12) Patent Application Publication
(10) Pub. No.: US 2026/0048026 A1
(43) Pub. Date: Feb. 19, 2026

(54) METHOD FOR EXTRACTION AND PREPARATION OF CURCUMIN

(71) Applicant: Shaanxi Undersun Biomedtech Co.,Ltd, Xi’an (CN)

(72) Inventors: GAOYAO HOU, Xi’an (CN); YINJUN HOU, Xi’an (CN)

(21) Appl. No.: 19/011,488

(22) Filed: Jan. 6, 2025

(30) Foreign Application Priority Data
Aug. 19, 2024 (CN) 202411132874.2

(51) Int. Cl. A61K 31/12; A61K 35/646; A61K 35/65; A61K 36/068; A61K 36/074; A61K 36/13; A61K 36/232; A61K 36/258; A61K 36/355; A61K 36/539; A61K 36/748; A61K 36/899; A61K 36/9066; A61K 47/10; A61K 47/42

(52) U.S. Cl. CPC A61K 31/12; A61K 35/646; A61K 35/65; A61K 36/068; A61K 36/074; A61K 36/13; A61K 36/232; A61K 36/258; A61K 36/355; A61K 36/539; A61K 36/748; A61K 36/899; A61K 36/9066; A61K 47/10; A61K 47/42; A61K 2236/15; A61K 2236/331

(57) ABSTRACT

The present invention discloses a method for extraction and preparation of curcumin, and in particular relates to the technical field of preparation of curcumin, which comprises the following steps: S1˜S9. For the present invention, the preparation process is simple, in which the curcumin can be fully utilized, the waste of raw materials can be minimized, and the nutritional elements of curcumin may be enriched to enhance its efficacy safely without toxic or side effect.

### CROSS-REFERENCE TO RELATED APPLICATION

This application claims priority of Chinese Patent Application No. 202411132874.2, filed on Aug. 19, 2024, the entire contents of which are incorporated herein by reference.

### TECHNICAL FIELD

The present invention relates to the technical field of preparation of curcumin, and more specifically to a method for extraction and preparation of curcumin.

### BACKGROUND

Curcumin, a natural yellow phenolic compound extracted from plants of Zingiberaceae or Araceae, is an orange yellow crystalline powder, slightly bitter and insoluble in water. At present, it is widely used in industries like medicine, food, feed and dyeing. The curcumin contains three main ingredients, i.e., curcumin (C₂₁H₂₀O₆, molecular weight 368, about 70%), demethoxycurcumin (C₂₀H₁₈O₅, molecular weight 338, about 15%) and bisdemethoxycurcumin (C₁₉H₁₆O₄, molecular weight 308, about 10%). The three curcumin monomers are significantly different in anti-tumor activity, including the inhibitory effect on endothelial cell growth. In medical applications, as the most active one, bisdemethoxycurcumin has pharmacological effects of anti-oxidation, anti-cancer, free radical elimination, lipid lowering, and improvement of digestive system and cardiovascular system. Additionally, it may inhibit tumor growth and angiogenesis definitely in vivo, so it is expected to be a new type of anti-tumor traditional Chinese medicine.

At present, there are reports concerning the preparation method for extracting the active ingredients of curcumin from rhizoma Curcuma longae. For example, the Chinese Patent (CN107715052A) discloses a method of preparing total curcumin by neutral alumina chromatography column with low carbon alcohol, ethyl acetate and acetone as the solvents. The Chinese Patent (CN103193610A) discloses a method for extracting, purifying and preparing curcumin, demethoxycurcumin and bisdemethoxycurcumin by multi-stage countercurrent extraction and macroporous resin column chromatography respectively based on alcohol-amino acid water. The Chinese Patent (103570517A) discloses a method for preparing curcumin based on alkaline water extraction, organic solvent de-esterification and acidification. The technological processes mentioned above are relatively complex and not suitable for industrialization for large amount of solvents and high production cost.

Moreover, the turmeric residue after curcumin extraction treated as waste cannot be fully utilized. In addition, the nutritional elements of curcumin cannot be enriched, and thus its efficacy cannot be enhanced.

In order to solve the above problems, a technical scheme is provided.

### SUMMARY

To overcome the above-mentioned defects in the prior art, an embodiment for the present invention has provided a method for extraction and preparation of curcumin to solve the problems raised in the above background techniques.

To attain the above-mentioned object, the present invention provides the following technical scheme:

A method for extraction and preparation of curcumin, comprising the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution; Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

According to one preferred embodiment, in Step S1, the temperature of the oven is set to 60-80° C., the drying time is set to 40-60 min, and during the drying process, the rhizome of turmeric plant is turned over once every 10 min.

According to one preferred embodiment, in Step S2, after grinding, the powder of the rhizome of turmeric plant is sifted with 200-mesh screen.

According to one preferred embodiment, in Step S3, the stirring speed is set to 200-300 r/min, and the heating temperature of the water bath is set to 70-80° C.

According to one preferred embodiment, in Step S4, the evaporation rate of the concentration process is 200-400 L/m²·h, and after concentration, the residual solvent is less than 3,000 ppm.

According to one preferred embodiment, in Step S5, two (upper and lower) layers of filters are used for washing and filtration, and the aperture for the lower layer is smaller than that for the upper layer.

According to one preferred embodiment, in Step S6, the rotary evaporator is heated to 80-90° C. for removing the solvent until the liquid crystal is precipitated.

According to one preferred embodiment, in Step S8, 5-8 parts of waste residue powder are adopted, and at the same time, 12-16 parts of purified water, 10-15 parts of glycerin, 14-19 parts of gelatin, 12-16 parts of Ganoderma lucidum, 22-26 parts of taxus, 17-22 parts of toad, 6-10 parts of whole scorpion, 12-16 parts of cordyceps sinensis, 20-26 parts of American ginseng, 18-22 parts of angelica sinensis, 4-8 parts of panax notoginseng, 13-18 parts of honeysuckle, 9-16 parts of spreading hedyotis herb, 11-15 parts of scutellariae barbatae and 10-14 parts of lalang grass rhizome are prepared;

Ganoderma lucidum, taxus, toad, whole scorpion, cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient;

The waste residue powder, product powder, gelatin and purified water are stirred in a constant temperature stirrer to form glue solution;

The glue solution is further stirred after glycerin is added in it, and a viscous liquid is obtained;

The viscous liquid is poured on the template, which is then placed in the oven for drying, and thus, the crust is obtained;

The rotational speed of the pulverizer is set to 400-600 r/min, and the pulverizing time is set to 50-70 min. As excipient, the powder is passed through a 100-mesh sieve after pulverizing.

Ganoderma lucidum can tonify qi, calm nerves, suppress coughing and relieve asthma;

Taxus can induce diuresis, relieve swelling, warm kidney and promote menstruation, and it is used to treat symptoms like edema, kidney disease, and gonorrhea;

Toad can remove toxin, eliminate stagnation, draw out furuncle and relieve swelling, and its gallbladder can be used to suppress cough and eliminate phlegm;

The whole scorpion can dispel wind and relieve convulsion, dredge collaterals, relieve pain, counteract toxic substances and eliminate stagnation;

Cordyceps sinensis can tonify kidney, benefit lung, stop bleeding and reduce phlegm with indications of aching lumbus and knees, prolonged cough, deficient dyspnea, overstrain cough and bloody phlegm;

American ginseng, a bitter-cold herb for clearing, cold in nature and sweet in taste, may enter the heart, lung, and kidney meridians. As a cold tonic, it is good for not only boosting qi and nourishing yin, but also clearing the internal heat and promoting the secretion of saliva or body fluid. American ginseng is also used to treat cardiovascular diseases, enhance immunity, and inhibit the proliferation of cancer cells;

Angelica sinensis can replenish blood, promote blood circulation, regulate menstruation, relieve pain, moisten dryness and loosen bowel;

Panax notoginseng can replenish blood, resist inflammation, promote blood circulation, remove blood stasis, resist oxidation and aging, dilate blood vessels, reduce coronary resistance, increase coronary flow, strengthen or improve coronary microcirculation, augment nutritional myocardial blood flow, lower arterial pressure, slightly reduce heart rate, and thus lower myocardial oxygen consumption significantly;

Honeysuckle can not only dissipate wind and heat, but also clear blood poison. It has significant effects in treating various heat diseases, such as general fever, rash, macular eruption, heat toxin, abscess and carbuncle, and sore throat;

The main functions of spreading hedyotis herb include “clearing away heat and toxic materials, promoting blood circulation and relieving swelling, removing dampness and jaundice with indications of lung heat with dyspnea and cough, pulmonary abscess, sore throat, intestinal carbuncle, boils and swollen sores, poisonous snake bites, heat strangury and stranguria, edema, dysentery and enteritis, damp heat jaundice, and cancerous swelling”;

Scutellariae barbatae can clear away heat and toxic materials, activate blood circulation, remove stasis, relieve swelling and alleviate pain, and resist cancer;

Lalang grass rhizome is indicated for blood heat, hematemesis, epistaxis, hematuria, polydipsia for fever, cough with lung heat, stomach heat and vomiting, jaundice due to damp heat, edema and oliguria, heat strangury and stranguria.

According to one preferred embodiment, the temperature of the constant temperature stirrer is set to 50-60° C., and the stirring time is set to 30-40 min.

According to one preferred embodiment, the thickness of the crust is set to 1 mm, the temperature of the oven is set to 35-40° C., and the drying time is set to 10-14 h.

Technical effects and advantages of the method for extraction and preparation of curcumin for the present invention:

In the present invention, the rhizome of turmeric plant is adopted, its powder is collected by sifting with 60-mesh screen after relevant treatment, and then crude product powder is obtained by soaking, heating in a water bath, stirring, concentration, washing, crystallization and pulverizing. The preparation process is simple.

The present invention can utilize waste residue of curcumin to make the crust for the crude product powder, thus taking full advantage of curcumin and minimizing the waste of raw materials.

The present invention can add Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome to the waste residue of curcumin, enrich the nutritional elements of curcumin, and enhance the efficacy of the product safely without toxic or side effect. The product can be used for anti-oxidation, anti-virus, liver protection, choleretic, anti-cancer, hypolipidemic, anti-HIV, bacteriostatic agent, etc. Compared with curcumin itself, the product can not only greatly improve the stability and bioavailability of curcumin, but also evidently advance the cytotoxicity and anti-cancer ability to cancer cells of curcumin.

### BRIEF DESCRIPTION OF THE DRAWINGS

FIG. 1 is a schematic diagram illustrating the method for extraction and preparation of curcumin for the present invention.

### DETAILED DESCRIPTION OF THE EMBODIMENTS

Hereunder the technical scheme in the embodiments of the present invention will be described clearly and completely with reference to the accompanying drawings of the embodiments. Obviously, the embodiments described herein are only some embodiments of the present invention, but not all possible embodiments of the present invention. Those skilled in the art can obtain other embodiments based on the embodiments provided herein without expending any creative labor; however, all those embodiments shall be deemed as falling in the scope of protection for the present invention.

### Embodiment 1

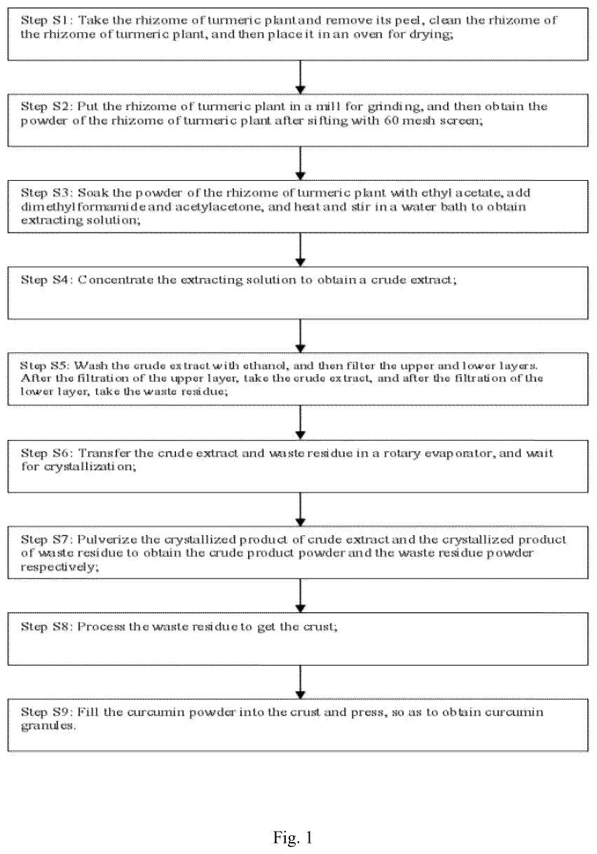

FIG. 1 provides the method for extraction and preparation of curcumin for the present invention, which comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution; Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

In Step S1, the temperature of the oven is set to 60° C., the drying time is set to 40 min, and during the drying process, the rhizome of turmeric plant is turned over once every 10 min; in Step S2, after grinding, the powder of the rhizome of turmeric plant is sifted with 200-mesh screen; in Step S3, the stirring speed is set to 200 r/min, and the heating temperature of the water bath to is set 70° C.; in Step S4, the evaporation rate of the concentration process is 200 L/m²·h, and after concentration, the residual solvent is less than 3,000 ppm; in Step S5, two (upper and lower) layers of filters are used for washing and filtration, and the aperture for the lower layer is smaller than that for the upper layer; in Step S6, the rotary evaporator is heated to 80° C. for removing the solvent until the liquid crystal is precipitated; in Step S8, 5 parts of waste residue powder are adopted, and at the same time, 12 parts of purified water, 10 parts of glycerin, 14 parts of gelatin, 12 parts of Ganoderma lucidum, 22 parts of taxus, 17 parts of toad, 6 parts of whole scorpion, 12 parts of Cordyceps sinensis, 20 parts of American ginseng, 18 parts of angelica sinensis, 4 parts of panax notoginseng, 13 parts of honeysuckle, 9 parts of spreading hedyotis herb, 11 parts of scutellariae barbatae and 10 parts of lalang grass rhizome are prepared. Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient. The waste residue powder, product powder, gelatin and purified water are stirred in a constant temperature stirrer to form glue solution. The glue solution is further stirred after glycerin is added in it, and a viscous liquid is obtained. The viscous liquid is poured on the template, which is then placed in the oven for drying, and thus, the crust is obtained. The rotational speed of the pulverizer is set to 400 r/min, and the pulverizing time is set to 50 min. As excipient, the powder is passed through a 100-mesh sieve after pulverizing. The temperature of the constant temperature stirrer is set to 50° C., and the stirring time is set to 30 min. The thickness of the crust is set to 1 mm, the temperature of the oven is set to 35° C., and the drying time is set to 10 h.

### Embodiment 2

FIG. 1 provides the method for extraction and preparation of curcumin for the present invention, which comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution;
Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

In Step S1, the temperature of the oven is set to 65° C., the drying time is set to 45 min, and during the drying process, the rhizome of turmeric plant is turned over once every 10 min; in Step S2, after grinding, the powder of the rhizome of turmeric plant is sifted with 200-mesh screen; in Step S3, the stirring speed is set to 220 r/min, and the heating temperature of the water bath to 72° C.; in Step S4, the evaporation rate of the concentration process is 250 L/m²·h, and after concentration, the residual solvent is less than 3,000 ppm; in Step S5, two (upper and lower) layers of filters are used for washing and filtration, and the aperture for the lower layer is smaller than that for the upper layer; in Step S6, the rotary evaporator is heated to 82° C. for removing the solvent until the liquid crystal is precipitated; in Step S8, 5 parts of waste residue powder are adopted, and at the same time, 13 parts of purified water, 11 parts of glycerin, 15 parts of gelatin, 13 parts of Ganoderma lucidum, 23 parts of taxus, 18 parts of toad, 7 parts of whole scorpion, 13 parts of Cordyceps sinensis, 21 parts of American ginseng, 19 parts of Angelica sinensis, 5 parts of Panax notoginseng, 14 parts of honeysuckle, 10 parts of spreading hedyotis herb, 12 parts of scutellariae barbatae and 11 parts of lalang grass rhizome are prepared. Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient. The waste residue powder, product powder, gelatin and purified water are stirred in a constant temperature stirrer to form glue solution. The glue solution is further stirred after glycerin is added in it, and a viscous liquid is obtained. The viscous liquid is poured on the template, which is then placed in the oven for drying, and thus, the crust is obtained. The rotational speed of the pulverizer is set to 450 r/min, and the pulverizing time is set to 55 min. As excipient, the powder is passed through a 100-mesh sieve after pulverizing. The temperature of the constant temperature stirrer is set to 52° C., and the stirring time is set to 32 min. The thickness of the crust is set to 1 mm, the temperature of the oven is set to 36° C., and the drying time is set to 11 h.

### Embodiment 3

FIG. 1 provides the method for extraction and preparation of curcumin for the present invention, which comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution;
Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

In Step S1, the temperature of the oven is set to 70° C., the drying time is set to 50 min, and during the drying process, the rhizome of turmeric plant is turned over once every 10 min; in Step S2, after grinding, the powder of the rhizome of turmeric plant is sifted with 200-mesh screen; in Step S3, the stirring speed is set to 250 r/min, and the heating temperature of the water bath is set to 75° C.; in Step S4, the evaporation rate of the concentration process is 300 L/m²·h, and after concentration, the residual solvent is less than 3,000 ppm; in Step S5, two (upper and lower) layers of filters are used for washing and filtration, and the aperture for the lower layer is smaller than that for the upper layer; in Step S6, the rotary evaporator is heated to 85° C. for removing the solvent until the liquid crystal is precipitated; in Step S8, 6 parts of waste residue powder are adopted, and at the same time, 14 parts of purified water, 13 parts of glycerin, 16 parts of gelatin, 14 parts of Ganoderma lucidum, 24 parts of taxus, 20 parts of toad, 8 parts of whole scorpion, 14 parts of Cordyceps sinensis, 24 parts of American ginseng, 20 parts of angelica sinensis, 6 parts of panax notoginseng, 15 parts of honeysuckle, 12 parts of spreading hedyotis herb, 13 parts of scutellariae barbatae and 12 parts of lalang grass rhizome are prepared. Ganoderma lucidum, taxus, toad, whole scorpion, cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient. The waste residue powder, product powder, gelatin and purified water are stirred in a constant temperature stirrer to form glue solution. The glue solution is further stirred after glycerin is added in it, and a viscous liquid is obtained. The viscous liquid is poured on the template, which is then placed in the oven for drying, and thus, the crust is obtained. The rotational speed of the pulverizer is set to 500 r/min, and the pulverizing time is set to 60 min. As excipient, the powder is passed through a 100-mesh sieve after pulverizing. The temperature of the constant temperature stirrer is set to 55° C., and the stirring time is set to 35 min. The thickness of the crust is set to 1 mm, the temperature of the oven is set to 38° C., and the drying time is set to 12 h.

### Embodiment 4

FIG. 1 provides the method for extraction and preparation of curcumin for the present invention, which comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution;
Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

In Step S1, the temperature of the oven is set to 75° C., the drying time is set to 55 min, and during the drying process, the rhizome of turmeric plant is turned over once every 10 min; in Step S2, after grinding, the powder of the rhizome of turmeric plant is sifted with 200-mesh screen; in Step S3, the stirring speed is set to 280 r/min, and the heating temperature of the water bath is set to 78° C.; in Step S4, the evaporation rate of the concentration process is 350 L/m²·h, and after concentration, the residual solvent is less than 3,000 ppm; in Step S5, two (upper and lower) layers of filters are used for washing and filtration, and the aperture for the lower layer is smaller than that for the upper layer; in Step S6, the rotary evaporator is heated to 88° C. for removing the solvent until the liquid crystal is precipitated; in Step S8, 7 parts of waste residue powder are adopted, and at the same time, 15 parts of purified water, 14 parts of glycerin, 18 parts of gelatin, 15 parts of Ganoderma lucidum, 25 parts of taxus, 21 parts of toad, 9 parts of whole scorpion, 15 parts of Cordyceps sinensis, 25 parts of American ginseng, 21 parts of angelica sinensis, 7 parts of panax notoginseng, 17 parts of honeysuckle, 15 parts of spreading hedyotis herb, 14 parts of scutellariae barbatae and 13 parts of lalang grass rhizome are prepared. Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient. The waste residue powder, product powder, gelatin and purified water are stirred in a constant temperature stirrer to form glue solution. The glue solution is further stirred after glycerin is added in it, and a viscous liquid is obtained. The viscous liquid is poured on the template, which is then placed in the oven for drying, and thus, the crust is obtained. The rotational speed of the pulverizer is set to 550 r/min, and the pulverizing time is set to 65 min. As excipient, the powder is passed through a 100-mesh sieve after pulverizing. The temperature of the constant temperature stirrer is set to 58° C., and the stirring time is set to 38 min. The thickness of the crust is set to 1 mm, the temperature of the oven is set to 39° C., and the drying time is set to 13 h.

### Embodiment 5

FIG. 1 provides the method for extraction and preparation of curcumin for the present invention, which comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution;
Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

In Step S1, the temperature of the oven is set to 80° C., the drying time is set to 60 min, and during the drying process, the rhizome of turmeric plant is turned over once every 10 min; in Step S2, after grinding, the powder of the rhizome of turmeric plant is sifted with 200-mesh screen; in Step S3, the stirring speed is set to 300 r/min, and the heating temperature of the water bath is set to 80° C.; in Step S4, the evaporation rate of the concentration process is 400 L/m²·h, and after concentration, the residual solvent is less than 3,000 ppm; in Step S5, two (upper and lower) layers of filters are used for washing and filtration, and the aperture for the lower layer is smaller than that for the upper layer; in Step S6, the rotary evaporator is heated to 90° C. for removing the solvent until the liquid crystal is precipitated; in Step S8, 8 parts of waste residue powder are adopted, and at the same time, 16 parts of purified water, 15 parts of glycerin, 19 parts of gelatin, 16 parts of Ganoderma lucidum, 26 parts of taxus, 22 parts of toad, 10 parts of whole scorpion, 16 parts of Cordyceps sinensis, 26 parts of American ginseng, 22 parts of angelica sinensis, 8 parts of panax notoginseng, 18 parts of honeysuckle, 16 parts of spreading hedyotis herb, 15 parts of scutellariae barbatae and 14 parts of lalang grass rhizome are prepared. Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient. The waste residue powder, product powder, gelatin and purified water are stirred in a constant temperature stirrer to form glue solution. The glue solution is further stirred after glycerin is added in it, and a viscous liquid is obtained. The viscous liquid is poured on the template, which is then placed in the oven for drying, and thus, the crust is obtained. The rotational speed of the pulverizer is set to 600 r/min, and the pulverizing time is set to 70 min. As excipient, the powder is passed through a 100-mesh sieve after pulverizing. The temperature of the constant temperature stirrer is set to 60° C., and the stirring time is set to 40 min. The thickness of the crust is set to 1 mm, the temperature of the oven is set to 40° C., and the drying time is set to 14 h.

The study included 30 male rats, body weight: 160-180 g. After 5 days of acclimation by feeding, the animals were stratified according to body weight based on a random number table and randomized into 6 groups, 5 animals each group. The ingredients of high-fat feed for the animals are as follows: lard 10%, cholesterol 2%, propylthiouracil 0.2%, pig bile salt 0.5%, and basal feed 87.3%. Five groups were given curcumin prepared in Embodiments 1-5 with high-fat feed and one group was given curcumin powder (as a comparative example) with high-fat feed for 19 consecutive days. On the 20th day, all of the rats were anesthetized with 25% urethan (0.4 ml/100 g), and their blood was collected through the abdominal aorta, and centrifuged for separating the serum, which was used for determining TG (mmol/L) and CHO (mmol/L). The results are as shown in the following table:

| | TG (mmol/L) | CHO (mmol/L) |
|---|---|---|
| Embodiment 1 | 0.52 | 4.6 |
| Embodiment 2 | 0.48 | 4.8 |
| Embodiment 3 | 0.41 | 4.2 |
| Embodiment 4 | 0.49 | 4.7 |
| Embodiment 5 | 0.44 | 4.6 |
| Comparative example | 0.58 | 5.2 |

The above results have clearly shown that the crust prepared by recycling the waste residue of curcumin and using Ganoderma lucidum, taxus, toad, whole scorpion, Cordyceps sinensis, American ginseng, angelica sinensis, panax notoginseng, honeysuckle, spreading hedyotis herb, scutellariae barbatae and lalang grass rhizome can enhance the therapeutic effect on diseases like cardiovascular disorder, and Embodiment 3 is the best one.

The above are only some embodiments of the present application, but the scope of protection for the present application is not limited to this. That is, any change or replacement that those skilled in the art can easily think of within the scope of the prior art disclosed in the present application shall be covered by the scope of protection for the present application. Therefore, the scope of protection for the patent application is only defined by the annexed claims.

Finally, the above are only preferred embodiments of the present invention, which are not intended to limit the present invention, any modification, equivalent replacement, or refinement to the embodiments without departing from the spirit and principle of the present invention shall be deemed as falling into the scope of protection for the present invention.

1. A method for extraction and preparation of curcumin, characterized in that the method comprises the following steps:
Step S1: Take the rhizome of turmeric plant and remove its peel, clean the rhizome of turmeric plant, and then place it in an oven for drying;
Step S2: Put the rhizome of turmeric plant in a mill for grinding, and then obtain the powder of the rhizome of turmeric plant after sifting with 60 mesh screen;
Step S3: Soak the powder of the rhizome of turmeric plant with ethyl acetate, add dimethylformamide and acetylacetone, and heat and stir in a water bath to obtain extracting solution;
Step S4: Concentrate the extracting solution to obtain a crude extract;
Step S5: Wash the crude extract with ethanol, and then filter the upper and lower layers. After the filtration of the upper layer, take the crude extract, and after the filtration of the lower layer, take the waste residue;
Step S6: Transfer the crude extract and waste residue in a rotary evaporator, and wait for crystallization;
Step S7: Pulverize the crystallized product of crude extract and the crystallized product of waste residue to obtain the crude product powder and the waste residue powder respectively;
Step S8: Process the waste residue to get the crust;
Step S9: Fill the crude product powder into the crust and press, so as to obtain curcumin granules.

2. The method for extraction and preparation of curcumin according to claim 1, characterized in that:
In Step S1, the temperature of the oven is set to 60-80° C., the drying time is set to 40-60 min, and during the drying process, the rhizome of turmeric plant is turned over once every 10 min.

3. The method for extraction and preparation of curcumin according to claim 1, characterized in that:
In Step S2, after grinding, the powder of the rhizome of turmeric plant is sifted with 200-mesh screen.

4. The method for extraction and preparation of curcumin according to claim 1, characterized in that:
In Step S3, the stirring speed is set to 200-300 r/min, and the heating temperature of the water bath is set to 70-80° C.

5. The method for extraction and preparation of curcumin according to claim 1, characterized in that:
In Step S4, the evaporation rate of the concentration process is 200-400 L/m²·h, and after concentration, the residual solvent is less than 3,000 ppm.

6. The method for extraction and preparation of curcumin according to claim 1, characterized in that:
In Step S5, two (upper and lower) layers of filters are used for washing and filtration, and the aperture for the lower layer is smaller than that for the upper layer.

7. The method for extraction and preparation of curcumin according to claim 1, characterized in that:
In Step S6, the rotary evaporator is heated to 80-90° C. for removing the solvent until the liquid crystal is precipitated.

8. The method for extraction and preparation of curcumin according to claim 1, characterized in that:
In Step S8, 5-8 parts of waste residue powder are adopted, and at the same time, 12-16 parts of purified water, 10-15 parts of glycerin, 14-19 parts of gelatin, 12-16 parts of Ganoderma lucidum, 22-26 parts of taxus, 17-22 parts of toad, 6-10 parts of whole scorpion, 12-16 parts of Cordyceps sinensis, 20-26 parts of American ginseng, 18-22 parts of angelica sinensis, 4-8 parts of panax notoginseng, 13-18 parts of honeysuckle, 9-16 parts of spreading hedyotis herb, 11-15 parts of scutellariae barbatae and 10-14 parts of lalang grass rhizome are prepared;
Ganoderma lucidum, taxus, toad, whole scorpion, cordyceps sinensis, American ginseng, angelica sinensis, panax notoginseng, honeysuckle, spreading hedyotis herb, scutellariae barbatae and lalang grass rhizome are added to a pulverizer, and pulverized to powder as excipient;
The waste residue powder, product powder, gelatin and purified water are stirred in a constant temperature stirrer to form glue solution;
The glue solution is further stirred after glycerin is added in it, and a viscous liquid is obtained;
The viscous liquid is poured on the template, which is then placed in the oven for drying, and thus, the crust is obtained;
The rotational speed of the pulverizer is set to 400-600 r/min, and the pulverizing time is set to 50-70 min. As excipient, the powder is passed through a 100-mesh sieve after pulverizing.

9. The method for extraction and preparation of curcumin according to claim 8, characterized in that:
The temperature of the constant temperature stirrer is set to 50-60° C., and the stirring time is set to 30-40 min.

10. The method for extraction and preparation of curcumin according to claim 8, characterized in that:
The thickness of the crust is set to 1 mm, the temperature of the oven is set to 35-40° C., and the drying time is set to 10-14 h.
